(19) United States
(12) Patent Application Publication
(10) Pub. No.: US 2010/0275933 A1
(43) Pub. Date: Nov. 4, 2010

(54) APPARATUS FOR SECURING A BODY TO AN IMMOBILIZATION BOARD

(75) Inventor: CLIFFORD A. YOUNG, San Jose, CA (US)

Correspondence Address: RAYMOND R. MOSER JR., ESQ.;MOSER IP LAW GROUP, 1030 BROAD STREET, SUITE 203, SHREWSBURY, NJ, 07702, US

(73) Assignee: CLIFFTECH, LLC., San Jose, CA (US)

(21) Appl. No.: 12/433,010

(22) Filed: Apr. 30, 2009

(51) Int. Cl. A61F 5/37

(52) U.S. Cl. 128/876

(57) ABSTRACT

An apparatus for securing a body to an immobilization board, comprising: a strap having a first end and a second end; and a device to movably couple the first end of the strap to the second end of the strap, wherein the device comprises a first lateral member coupled to the first end of the strap, a second lateral member for providing tension to the strap, a third lateral member having a corrugated side for applying friction to the strap.

### BACKGROUND

1. Field

The present invention generally relates to emergency medical devices and, more particularly, to an apparatus for securing a body to an immobilization board.

2. Description of the Related Art

High energy collisions, such as those sustained during car accidents, recreational activities, or sporting events occasionally result in significant injury, such as neck, back or head injuries. Generally, when treating a victim with a suspected head and/or neck injury, stabilization of the head and neck is a matter of high priority. Stabilization is accomplished by securing the victim to a rigid board (spine board, long board, backboard, half board, etc.) via a number of straps positioned across the victim to ensure stability. The straps typically consist of a mechanism or device to adjust the length and secure the strap to a desired position.

Fabric hook and loop fasteners (e.g., VELCRO®) are commonly used to secure the strap to the desired position. However, fabric hook and loop fasteners do not provide the ability to make accurate adjustments necessary to ensure the victim is properly secured to the rigid board. D or O-ring attachments may be used in place of, or in conjunction with, hook and loop fasteners to allow the user to make adjustments. However, a D or O-ring attachment does not adequately prevent the strap from slipping out of position. Another commonly used device is a seat belt—type mechanical buckle. However, such buckles consist of moving parts which are susceptible to wear, leading to degradation and possible failure. The above mentioned deficiencies in the currently used devices do not provide an adequate means for reliably securing the victim to the board, thus increasing the risk of the victim falling off of the board and sustaining further injuries.

Therefore, there is a need in the art for an improved apparatus for securing a body to an immobilization board.

### SUMMARY

Embodiments of the present invention comprise an apparatus for securing a body to an immobilization board, comprising: a strap having a first end and a second end; and a device to movably couple the first end of the strap to the second end of the strap, wherein the device comprises a first lateral member coupled to the first end of the strap, a second lateral member for providing tension to the strap, a third lateral member having a corrugated side for applying friction to the strap.

In some embodiments, an apparatus for securing a body to an immobilization board comprises: a strap having a first end and a second end; and a tension lock buckle, for movably coupling the first end to the second end of the strap, wherein the tension lock buckle comprises a plurality of teeth for applying friction to the strap.

In some embodiments, an apparatus for securing a body to an immobilization board comprises: a board for supporting a body; a strap, having a first and second end, coupled to the board; and a device to movably couple the first end of the strap to the second end of the strap, wherein the device comprises a first lateral member coupled to the first end of the strap, a second lateral member for providing tension to the strap, a third lateral member having a corrugated side for applying friction to the strap.

### BRIEF DESCRIPTION OF THE DRAWINGS

Embodiments of the present invention, briefly summarized above and discussed in greater detail below, can be understood by reference to the illustrative embodiments of the invention depicted in the appended drawings. It is to be noted, however, that the appended figures illustrate only exemplary embodiments of this invention and are therefore not to be considered limiting of its scope, for the invention may admit to other equally effective embodiments.

FIG. 1 depicts a schematic view of an exemplary immobilization board suitable to be used with an apparatus for securing a body to an immobilization board in accordance with some embodiments of the present invention.

FIG. 2 depicts a top view of an apparatus for securing a body to an immobilization board in accordance with some embodiments of the present invention.

FIG. 3 depicts a bottom view of an apparatus for securing a body to an immobilization board in accordance with some embodiments of the present invention.

FIG. 4 depicts a front view of an apparatus for securing a body to an immobilization board in accordance with some embodiments of the present invention.

FIG. 5 depicts a side view of an apparatus for securing a body to an immobilization board in accordance with some embodiments of the present invention.

FIGS. 6 and 7 depict a side view of an apparatus for securing a body to an immobilization board in operation in accordance with some embodiments of the present invention.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present invention generally relate to emergency medical devices. The inventive apparatus includes a cervical spine strap for securing a body to an immobilization board. The inventive apparatus advantageously allows for precise, quick adjustments and is not susceptible to slipping or degradation due to break down of moving parts.

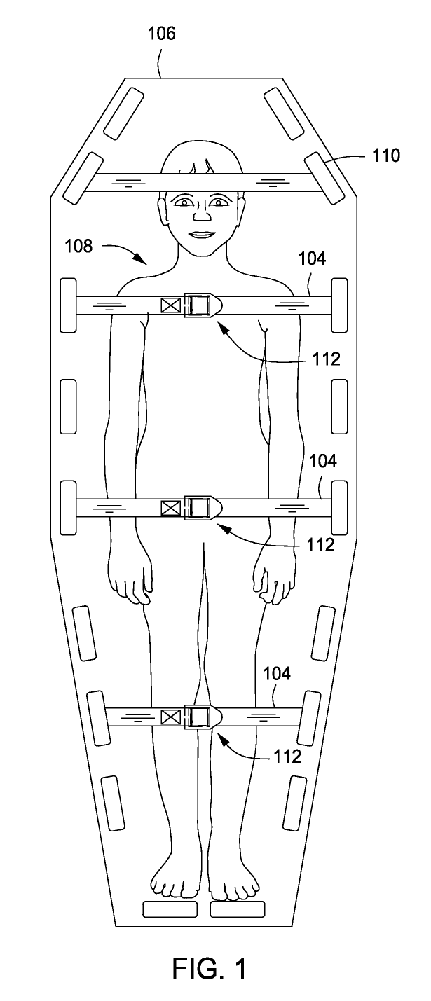

FIG. 1 depicts a schematic view of an exemplary immobilization board suitable to be used with an apparatus for securing a body to an immobilization board in accordance with some embodiments of the present invention. The immobilization board 106 generally comprises a rigid material for supporting a body 108. The rigid material may be any material suitable to provide support for the body 108. For example, the immobilization board 106 may comprise wood, plastic, or the like. In some embodiments, the material may be light weight so as to allow the immobilization board 106 to be easily maneuvered by an operator. In some embodiments, for example, where the immobilization board 106 may be used in an environment where there is water present (e.g., a beach or pool), the immobilization board 106 may comprise a material that is buoyant in water.

The shape and length of the immobilization board 106 may vary in accordance with the purpose of use, or the environment in which the immobilization board 106 may be used. In some embodiments, for example, in embodiments such as depicted in FIG. 1, the immobilization board 106 may be of a sufficient length that an entire body 108 may be supported. In some embodiments, the immobilization board 106 may be of a shorter length, for example, such that only the neck and head is supported, or such that the neck, head, and back are supported.

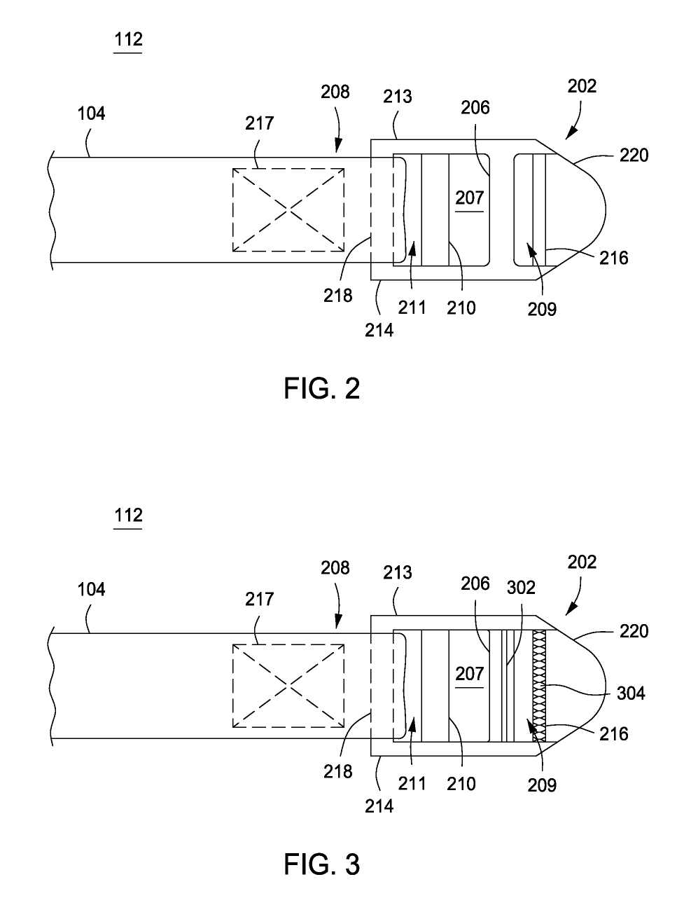

The apparatus 112 for securing a body 108 to the immobilization board 106, described below with respect to FIGS. 2-7, may be positioned in any location on the immobilization board 106 necessary to properly secure the body 108. In some embodiments, such as depicted in FIG. 1, more than one apparatus 112 (three shown) may be utilized. In such embodiments, the more than one apparatus 112 may be positioned at various locations of the immobilization board 106 to ensure a proper stabilization of the body 108. For example, each of the more than one apparatus 112 may be positioned parallel to one another at various locations of the immobilization board 106 or may be positioned to cross one another at various locations of the immobilization board 106.

In some embodiments, the immobilization board 106 comprises one or more slots or holes 110. The shape and amount of slots or holes 110 may be varied in accordance with the intended use of immobilization board 106. For example, in some embodiments, the slots or holes 110 may be positioned to allow for one or more operators to securely hold and carry the immobilization board 106. In some embodiments, the slots or holes 110 may be configured to allow the strap 104 of apparatus 112 to be fed through to prevent the strap 104 from moving out of a desired position during use of the immobilization board 106.

In some embodiments, the board 106 may comprise an additional support (not shown), for example, a cervical collar, neck brace, neck collar, or the like, to ensure the head and neck of the body 108 remains in a fixed position.

FIG. 2 depicts a top view of an apparatus 112 for securing a body 108 to an immobilization board 106 in accordance with some embodiments of the present invention.

The apparatus 112 generally comprises a strap 104 and a buckle 202. The strap 104 may comprise any suitable material capable of withstanding a sufficient amount of tension to ensure a body is properly secured to an immobilization board. For example, the strap 104 may have a breaking strength of up to about 860 lbs. The strap 104 may be any length suitable to reach around a body and an immobilization board to facilitate a secure fit. In some embodiments, the strap 104 may be up to about 9 ft. long.

The strap 104 may also comprise additional properties to ensure prolonged continuous use and reliability. For example, the strap 104 may be resistant to tear, stretching and absorption. In some embodiments, the strap 104 may be constructed from a material comprising a polymer, such as nylon, polypropylene, polyester, or the like. In some embodiments the strap 104 may be constructed from the material in a woven design, such as a webbing, cross stitching, braiding, or the like.

The buckle 202 generally comprises a first side 213, a second side 214, a first lateral arm 218 for securing a first end 208 of the strap 104, a second lateral arm 206 for applying tension to the strap 104, and a tab 220 having an extension 216. In some embodiments the buckle may comprise one or more additional lateral arms (one shown) 210 to increase the overall strength of the buckle 202 and provide an increased resistance to twisting. Between the lateral arms 218, 210, 206, and the tab 220 are spaces 211, 207, 209 to allow for the strap 104 to pass through. The first end 208 of the strap 104 may be looped around the first lateral arm 218 and secured via at least one of stitching 217, adhesive (not shown), rivets (not shown), staples (not shown), or the like.

The buckle 202 may be constructed from any suitable material capable of withstanding the forces associated with securing a body to an immobilization board. The material may also comprise one or more other beneficial properties, for example, resistance to absorption, abrasion, mold, chemical degradation, and the like. In some embodiments, the buckle 202 is constructed from a plastic or polymer, such as, polyacetal, acetal resin, polytrioxane, polyformaldehyde, polyoxymethylene (e.g., DELRIN®), nylon, polyvinyl chloride, or the like.

FIG. 3 depicts a bottom view of an apparatus 112 for securing a body to an immobilization board in accordance with some embodiments of the present invention. As can be seen in the figure, the second lateral arm 206 may be tapered to a bottom surface 302 having a smaller width than the top of the second lateral arm 206 to facilitate smooth movement of the strap 104. The second lateral arm 206 may be any shape sufficient as to not impede movement of the strap 104. For example, the second lateral arm 206 may be rounded, trapezoidal, triangular, or the like.

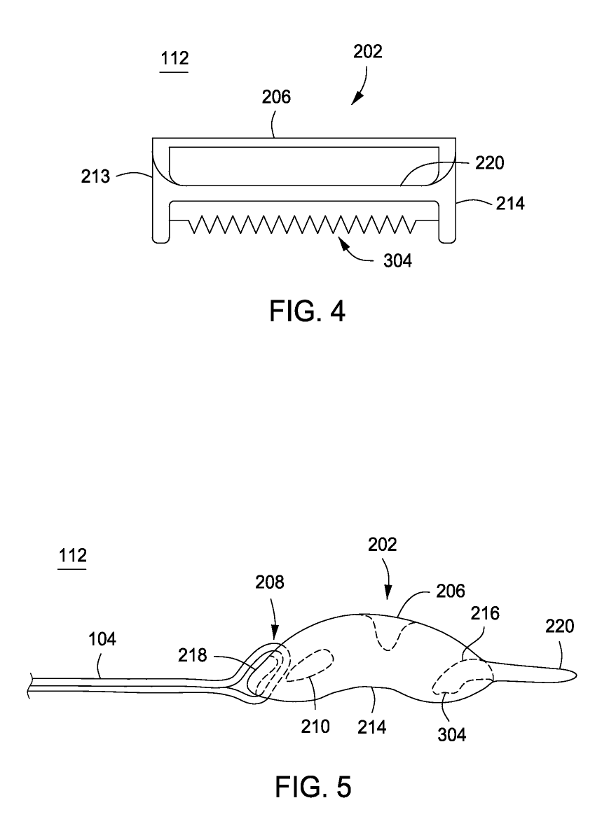

The bottom surface of extension 216 of tab 220 comprises a corrugated surface 304 to provide a frictional force to the strap 104, thus keeping it in a desired position. The corrugated surface 304 may comprise a plurality of elements having any shape sufficient to provide enough friction to the strap 104 to eliminate the possibility of the strap 104 slipping out of the desired position. For example, in some embodiments, such as the embodiment depicted in FIG. 4, the corrugated surface 304 may comprise a plurality of “teeth”.

As can be seen in the side view, depicted in FIG. 5, the sides 214 (212 not shown) of the buckle 202 may comprise a curved shape to conform to the surface of a body, facilitate comfort for the body being secured, and to aid in lengthening or removal of the strap via the tab 220.

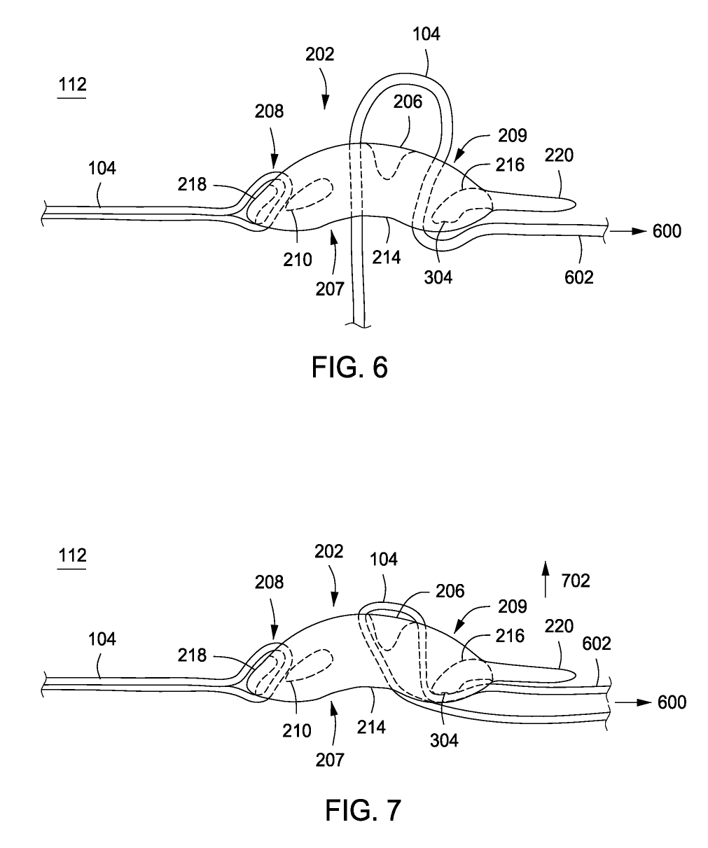

FIGS. 6 and 7 depict a side view of an apparatus for securing a body to an immobilization board in operation in accordance with some embodiments of the present invention.

Referring to FIG. 6, the second end 602 of the strap 104 is passed through the space 207 between lateral arm 210 and the second lateral arm 206. The second end 602 of the strap 104 is then looped around the second lateral arm 206 and passed back through space 209 between the second lateral arm 206 and the extension 216 of tab 220, and pulled through in a horizontal direction 600, passing under the corrugated surface 304 of tab extension 216.

Referring to FIG. 7, when the strap 104 is pulled in a horizontal direction 600 away from the buckle 202, the top surface of the strap 104 engages the corrugated surface 304 causing a frictional force sufficient to keep the strap 104 in a static position. To make adjustments or remove the apparatus 112, a user applies a force in an upward direction 702 to the tab 220, which causes the strap 104 to disengage from the corrugated surface 304, thereby releasing the friction and allowing the strap 104 to move freely.

While the foregoing is directed to embodiments of the present invention, other and further embodiments of the invention may be devised without departing from the basic scope thereof, and the scope thereof is determined by the claims that follow.

1. An apparatus for securing a body to an immobilization board, comprising:
a strap having a first end and a second end; and
a device to movably couple the first end of the strap to the second end of the strap, wherein the device comprises a first lateral member coupled to the first end of the strap, a second lateral member for providing tension to the strap, a third lateral member having a corrugated side for applying friction to the strap.

2. The apparatus of claim 1, wherein the device further comprises a tab for releasing friction applied to the strap.

3. The apparatus of claim 1, wherein the corrugated side comprises a plurality of teeth.

4. The apparatus of claim 1, wherein the strap comprises a webbing, cross stitched or braided material.

5. The apparatus of claim 4, wherein the material comprises at least one of polypropylene, polyester, or nylon.

6. The apparatus of claim 1, wherein the device comprises a plastic.

7. The apparatus of claim 4, wherein the plastic is one of polyacetal, acetal resin, polytrioxane, polyformaldehyde, polyoxymethylene, nylon, or polyvinyl chloride.

8. An apparatus for securing a body to an immobilization board, comprising:
a strap having a first end and a second end; and
a tension lock buckle for movably coupling the first end to the second end of the strap, wherein the tension lock buckle comprises a plurality of teeth for applying friction to the strap.

9. The apparatus of claim 8, wherein the tension lock buckle further comprises:
a first lateral member coupled to the first end of the strap;
a second lateral member for providing tension to the strap; and
a third lateral member having a corrugated side for applying friction to the strap.

10. The apparatus of claim 9, wherein the tension lock buckle further comprises a tab for releasing friction applied to the strap.

11. The apparatus of claim 9, wherein the corrugated surface comprises a plurality of teeth.

12. The apparatus of claim 9, wherein the strap comprises a webbing, cross stitched or braided material.

13. The apparatus of claim 12, wherein the material comprises at least one of polypropylene, polyester, or nylon.

14. The apparatus of claim 9, wherein the tension lock buckle comprises a plastic.

15. The apparatus of claim 14, wherein the plastic is one of polyacetal, acetal resin, polytrioxane, polyformaldehyde, polyoxymethylene, nylon, or polyvinyl chloride.

16. An apparatus for securing a body to an immobilization board, comprising:
a board for supporting a body;
a strap, having a first and second end, coupled to the board; and
a device to movably couple the first end of the strap to the second end of the strap, wherein the device comprises a first lateral member coupled to the first end of the strap, a second lateral member for providing tension to the strap, a third lateral member having a corrugated side for applying friction to the strap.

17. The apparatus of claim 16, wherein the device further comprises a tab for releasing friction applied to the strap.

18. The apparatus of claim 16, wherein the corrugated side comprises a plurality of teeth.

19. The apparatus of claim 16, wherein the strap comprises a webbing, cross stitched or braided material.

20. The apparatus of claim 16, wherein the device is a tension lock buckle.
